(19) United States
(12) Patent Application Publication
(10) Pub. No.: US 2008/0050444 A1
(43) Pub. Date: Feb. 28, 2008

(54) Pharmaceutical compositions of ilaparazole

(76) Inventors: Rajneesh Taneja, Libertyville, IL (US); Steve Berge, Shoreview, MN (US); Vijay Gupte, Vernon Hills, IL (US)

Correspondence Address: DYKEMA GOSSETT PLLC, 10 S. WACKER DR., STE. 2300, CHICAGO, IL, 60606, US

(21) Appl. No.: 11/639,866

(22) Filed: Dec. 15, 2006

Related U.S. Application Data
(60) Provisional application No. 60/750,968, filed on Dec. 16, 2005.

(51) Int. Cl. A61K 31/4439; A61K 31/4184; A61K 31/435; A61P 1/00; A61P 11/00; A61K 9/14; A61K 31/437

(52) U.S. Cl. 424/490; 424/489; 514/299; 514/303; 514/338; 514/395

(57) ABSTRACT

The present invention relates to pharmaceutical compositions comprising solid particles of an active ingredient that have a particle size of from about 0.1 micron to about 100 microns.

### RELATED APPLICATION INFORMATION

This application claims the benefit of U.S. Application No. 60/750,968 filed on Dec. 16, 2005, the contents of which are herein incorporated by reference.

### FIELD OF THE INVENTION

The present invention relates to pharmaceutical compositions comprising solid particles of an active ingredient that have a mean particle size from about 0.1 micron to about 100 microns. The present invention further relates to methods of treating gastrointestinal disorders or chronic cough in patients in need of treatment thereof using the pharmaceutical compositions of the present invention.

### BACKGROUND OF THE INVENTION

The stomach is an organ of digestion. It has a saclike shape and is located between the esophagus and the intestines. Almost every animal has a stomach.

The human stomach is a muscular, elastic, pear-shaped bag, lying crosswise in the abdominal cavity beneath the diaphragm. It changes size and shape according to its position within the body and the amount of food inside. The wall of the stomach is lined with millions of gastric glands, which together secrete 400-800 ml of gastric juice at each meal. Three kinds of cells are found in the gastric glands. These cells are parietal cells, “chief” cells and mucus-secreting cells. Parietal cells contain an enzyme known as H⁺/K⁺adenosine triphosphatase. H⁺/K⁺ adenosine triphosphatase is also referred to as an “acid pump” or “proton pump”. This transmembrane protein secretes H⁺ ions (protons) by active transport, using the energy of ATP. The concentration of H⁺ in the gastric juice can be as high as 0.15 M, giving gastric juice a pH less than 1.

Proton pump inhibitors (or “PPIs”) are a class of pharmaceutical compounds that inhibit gastric acid secretions by inhibiting H⁺/K⁺ adenosine triphosphatase. It is known in the art that proton pumps can exist in either an active state or a dormant state. PPIs only bind to the active proton pumps. PPIs are metabolized in the parietal cells to active sulfenamide metabolites that inactivate the sulfhydryl group of the proton pump, thereby reducing the hydrogen ion secretion (Langtry and Wilde, “An update of its pharmacological properties and clinical efficacy in the management of acid-related disorders,” Drugs, 54(3): 473-500 (1997)).

PPIs are frequently prescribed for short-term treatment of active duodenal ulcers, gastric ulcers, gastroesophageal reflux disease (GERD), severe erosive esophagitis, poorly responsive systematic GERD, and pathological hypersecretory conditions such as Zollinger Ellison syndrome. These conditions are caused by an imbalance between acid and pepsin production (aggressive factors), and mucous, bicarbonate and prostaglandin production (defensive factors). The above listed conditions can arise in healthy or critically ill patients, and may be accompanied by significant gastrointestinal bleeding.

2-[[(4-methoxy-3-methyl-2-pyridinyl)methyl]sulfinyl]-5-(1H-pyrrol-1-yl)-1H-benzimidazole, also known as ilaprazole, acts as a PPI. Methods for making ilaprazole are described in U.S. Pat. No. 5,703,097. It is known in the art that ilaprazole is unstable under acid or neutral conditions. In this regard, U.S. Pat. No. 6,280,773 describes a microgranule containing a 5-pyrrolyl-2-pyridylmethylsulfinylbenzimidaole derivative, such as ilaprazole, that is stabilized with an alkali compound.

There is a need in the art for pharmaceutical compositions containing PPIs, such as ilaprazole, that provide improved bioavailability and that exhibit a faster onset of action, particularly when compared to pharmaceutical compositions known in the art, such as the microgranules described in U.S. Pat. No. 6,280,773. Ilaprazole containing pharmaceutical compositions that exhibit improved bioavailability and that have a faster onset of action, would be particularly beneficial for patients suffering from gastrointestinal disorders, such as, symptomatic GERD, dyspepsia and heartburn.

### SUMMARY OF THE INVENTION

In one embodiment, the present invention relates to a pharmaceutical composition comprising an active ingredient, wherein said active ingredient has a mean particle size from about 0.1 micron to about 100 microns. The active ingredient that can be used in the composition can be a compound of the following formula I: 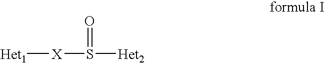
wherein Het₁ is 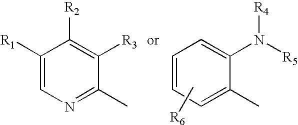
Het₂ is 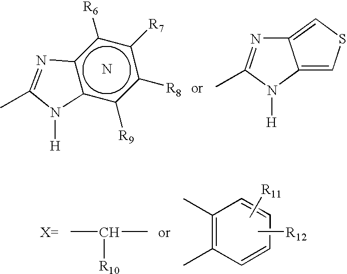
wherein

N in the benzimidazole moiety means that one of the ring carbon atoms substituted by R₆-R₉ optionally may be exchanged for a nitrogen atom without any substituents;

R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;

R₄ and R₅ are the same or different and selected from hydrogen, alkyl and arylalkyl;

R₆′ is hydrogen, halogen, trifluoromethyl, alkyl or alkoxy;

R₆-R₉ are the same or different and selected from hydrogen, alkyl, alkoxy, halogen, haloalkoxy, alkylcarbonyl, alkoxycarbonyl, oxazolinyl, trifluoroalkyl, a heterocyclic ring that may be further substituted or adjacent groups R₆-R₉ form ring structures which may be further substituted;

R₁₀ is hydrogen or forms an alkylene chain together with R₃ and R₁₁; and

R₁₂ are the same or different and selected from hydrogen, halogen or alkyl.

As mentioned above, the active ingredient in the pharmaceutical compositions of the present invention has a mean particle size of from about 0.1 micron to about 100 microns. More specifically, the active ingredient can have a mean particle size of from about 0.5 microns to about 75 microns. Preferably, the active ingredient has a particle size of from about 0.75 microns to about 65 microns. Even more preferably, the active ingredient has a particle size of from about 1 micron to about 50 microns.

Additionally, the present invention contemplates pharmaceutical compositions comprising solid particles having a particle size less than about 50 microns. More preferably, the pharmaceutical compositions of the present invention comprise solid particles of an active ingredient having a mean particle size of less than about 45 microns. And even more preferably, the pharmaceutical compositions of the present invention comprise solid particles of an active ingredient having a mean particle size of less than about 40 microns.

The pharmaceutical compositions of the present invention can contain a number of other ingredients in addition to the active ingredient, including but not limited to, at least one stabilizer, a surfactant, a coating, a binder, a glidant, a solubility enhancing agent, a sweetness and/or flavoring agent, a filler, lubricant, preservative, a buffer, a wetting agent, a humectant, an emulsifier, a preservative, an effervescent agent, a solution retarder, an absorption accelerator, a distintegrant or combinations of any of the above.

If the pharmaceutical composition of the present invention contains at least one stabilizer, said stabilizer can be a Group IA metal, a Group IIA metal, a bicarbonate salt of a Group IA metal, a bicarbonate salt of a Group IIA metal, a sodium salt, a magnesium salt, a calcium salt, an aluminum salt, a bicarbonate salt of magnesium, a bicarbonate salt of calcium, a bicarbonate salt of aluminum, polymers, sodium alginate, sterols, fatty alcohols or combinations of any of the above.

The pharmaceutical composition of the present invention can contain at least one enteric coating.

The pharmaceutical composition of the present invention exhibits site-specific absorption of the active ingredient. Therefore, the composition of the present invention, after absorption by a patient in need of treatment thereof, primarily releases the active ingredient in the area of the duodenum, the area of the upper jujenum or in a combination of the area of the duodenum and upper jujenum of said patient.

The pharmaceutical composition of the present invention can be in the form of a granule, microparticulate or microparticle. Granules, microparticulates or microparticles of the present invention can be placed into one or more capsules or compressed into tablets for administration to a patient in need of treatment thereof.

In a second embodiment, the present invention relates to a method of treating a gastrointestinal disorder. The method involves the step of administering to a patient in need of treatment thereof a therapeutically effective amount of the pharmaceutical composition described herein. Gastrointestinal disorders that can be treated using the hereinbefore described method include, but are not limited to, heartburn, inflammatory bowel disease, Crohn's disease, irritable bowel syndrome, ulcerative colitis, a peptic ulcer, a stress ulcer, a bleeding peptic ulcer, a duodenal ulcer, infectious enteritis, colitis, diverticulitis, gastric hyperacidity, dyspepsia, gastroparesis, Zollinger-Ellison syndrome, gastroesophageal reflux disease, Helicobacter pylori associated disease, short-bowel syndrome, hypersecretory states associated with systemic mastocytosis or basophilic leukemia or hyperhistaminemia, or combinations of any of the above disorders.

In a third embodiment, the present invention relates to a method of treating chronic cough in a patient in need of treatment thereof. The method involves the step of administering to a patient in need of treatment thereof a therapeutically effective amount of the pharmaceutical composition described herein.

In a fourth embodiment, the present invention relates to a pharmaceutical composition comprising an active ingredient, wherein said active ingredient has a mean particle size from about 0.1 micron to about 100 microns. The active ingredient that can be used in the composition is 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, or salts, metabolites, polymorphs, cocrystals or combinations of any of the above.

As mentioned above, the active ingredient in the pharmaceutical compositions of the present invention has a mean particle size of from about 0.1 micron to about 100 microns. More specifically, the active ingredient can have a mean particle size of from about 0.5 microns to about 75 microns. Preferably, the active ingredient has a particle size of from about 0.75 microns to about 65 microns. Even more preferably, the active ingredient has a particle size of from about 1 micron to about 50 microns.

Additionally, the present invention contemplates pharmaceutical compositions comprising solid particles having a particle size less than about 50 microns. More preferably, the pharmaceutical compositions of the present invention comprise solid particles of an active ingredient having a mean particle size of less than about 45 microns. And even more preferably, the pharmaceutical compositions of the present invention comprise solid particles of an active ingredient having a mean particle size of less than about 40 microns.

The pharmaceutical compositions of the present invention can contain a number of other ingredients in addition to the active ingredient, including but not limited to, at least one stabilizer, a surfactant, a coating, a binder, a glidant, a solubility enhancing agent, a sweetness and/or flavoring agent, a filler, lubricant, preservative, a buffer, a wetting agent, a humectant, an emulsifier, a preservative, an effervescent agent, a solution retarder, an absorption accelerator, a distintegrant or combinations of any of the above.

If the pharmaceutical composition of the present invention contains at least one stabilizer, said stabilizer can be a Group IA metal, a Group IIA metal, a bicarbonate salt of a Group IA metal, a bicarbonate salt of a Group IIA metal, a sodium salt, a magnesium salt, a calcium salt, an aluminum salt, a bicarbonate salt of magnesium, a bicarbonate salt of calcium, a bicarbonate salt of aluminum, polymers, sodium alginate, sterols, fatty alcohols or combinations of any of the above.

The pharmaceutical composition of the present invention can contain at least one enteric coating.

The pharmaceutical composition of the present invention exhibits site-specific absorption of the active ingredient. Therefore, the composition of the present invention, after absorption by a patient in need of treatment thereof, primarily releases the active ingredient in the area of the duodenum, the area of the upper jujenum or in a combination of the area of the duodenum and upper jujenum of said patient.

The pharmaceutical composition of the present invention can be in the form of a granule, microparticulate or microparticle. Granules, microparticulates or microparticles of the present invention can be placed into one or more capsules or compressed into tablets for administration to a patient in need of treatment thereof.

In addition, in the pharmaceutical composition of the present invention, at least 70%, at least 75%, at least 80%, at least 85% or at least 90% of the 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof is released from the composition within about 20 minutes when tested in an in vitro dissolution test Apparatus 1 (basket method) in 500 mL of a pH 7.5 buffer with about 0.5 sodium lauryl sulfate as the dissolution medium, at about 100 rpm and at a temperature of about 37° C.±0.5° C.

In a fifth embodiment, the present invention relates to a method of treating a gastrointestinal disorder. The method involves the step of administering to a patient in need of treatment thereof a therapeutically effective amount of the pharmaceutical composition described herein. Gastrointestinal disorders that can be treated using the hereinbefore described method include, but are not limited to, heartburn, inflammatory bowel disease, Crohn's disease, irritable bowel syndrome, ulcerative colitis, a peptic ulcer, a stress ulcer, a bleeding peptic ulcer, a duodenal ulcer, infectious enteritis, colitis, diverticulitis, gastric hyperacidity, dyspepsia, gastroparesis, Zollinger-Ellison syndrome, gastroesophageal reflux disease, Helicobacter pylori associated disease, short-bowel syndrome, hypersecretory states associated with systemic mastocytosis or basophilic leukemia or hyperhistaminemia, or combinations of any of the above disorders.

In a sixth embodiment, the present invention relates to a method of treating chronic cough in a patient in need of treatment thereof. The method involves the step of administering to a patient in need of treatment thereof a therapeutically effective amount of the pharmaceutical composition described herein.

### BRIEF DESCRIPTION OF THE FIGURES

FIG. 1 shows the particle size distribution of Lot A and Lot B of ilaprazole as described in Example 1.

FIG. 2 shows sugar sphere based ilaprazole formations (Formulation A) prepared pursuant to Example 2.

FIG. 3 shows Celphere CP305 based ilaprazole formulations (Formulation B) prepared pursuant to Example 2.

FIG. 4 shows the mean plasma concentration-time profiles for a single 10 mg dose of ilaprazole formulations A, B and C that are described in Example 3.

### DETAILED DESCRIPTION OF THE INVENTION

As used in this specification and the appended claims, the singular forms “a,” “an” and “the” include plural references unless the context clearly dictates otherwise. Thus, for example, reference to “an active agent” includes a single active agent as well two or more different active agents in combination.

In describing and claiming the present invention, the following terminology will be used in accordance with the definitions set out below.

The terms “active agent,” “active ingredient,” and “drug” are used interchangeably herein to refer to compounds of the general formula I (below), an alkaline salt thereof, a metabolite thereof or a prodrug thereof, one of the single enantiomers thereof, an alkaline salt thereof (such as, for example, Mg²⁺, Ca²⁺, Na⁺ or K⁺ salts), a metabolite thereof or a prodrug thereof or a single enantiomer of the compounds of the general formula I, an alkaline salt of a single enantiomer of compounds of the general formula I, a metabolite of a single enantiomer of compounds of general formula I or a prodrug of a single enantiomer of compounds of general formula I. 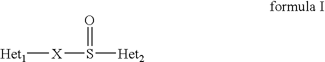
wherein Het₁ is 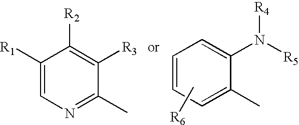
Het₂ is 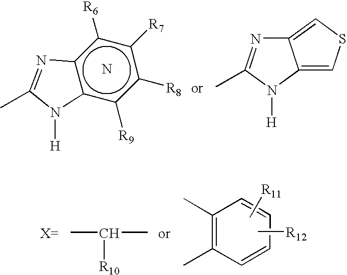
wherein

N in the benzimidazole moiety means that one of the ring carbon atoms substituted by R₆-R₉ optionally may be exchanged for a nitrogen atom without any substituents;

R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;

R₄ and R₅ are the same or different and selected from hydrogen, alkyl and arylalkyl;

R₆′ is hydrogen, halogen, trifluoromethyl, alkyl or alkoxy;

R₆-R₉ are the same or different and selected from hydrogen, alkyl, alkoxy, halogen, haloalkoxy, alkylcarbonyl, alkoxycarbonyl, oxazolinyl, trifluoroalkyl, a heterocyclic ring that may be further substituted or adjacent groups R₆-R₉ form ring structures which may be further substituted;

R₁₀ is hydrogen or forms an alkylene chain together with R₃ and R₁₁; and

R₁₂ are the same or different and selected from hydrogen, halogen or alkyl.

Preferred compounds according to formula I are:

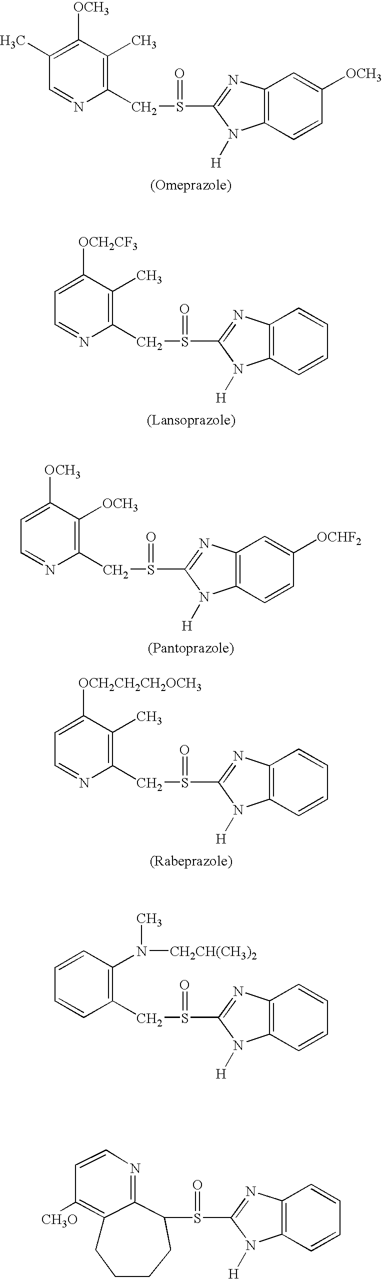

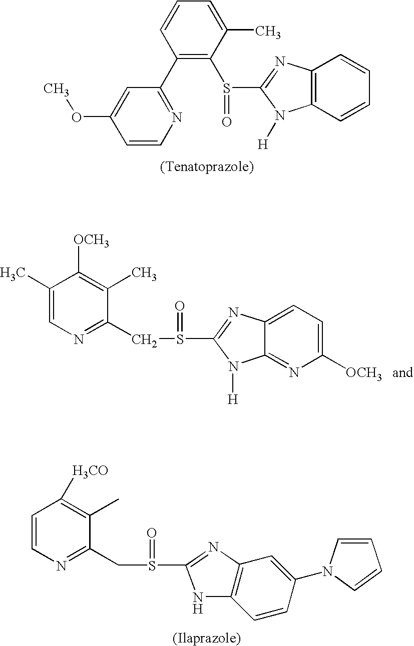

The most preferred compounds of formula I are: 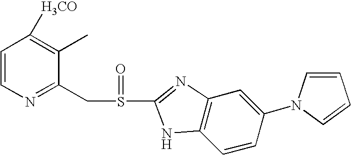
namely, 2-[[(4-methoxy-3-methyl)-2-pyridyl]methylsufinyl]-5-(1H-pyrrol-1-yl)benzimidazole, which, as mentioned above, is also known as “ilaprazole”, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts of 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole or (+)-2-[[(4-methoxy-3-methyl)-2-pyridyl]methylsufinyl]-5-(1H-pyrrol-1-yl)benzimidazole, metabolites of 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole or (+)-2-[[(4-methoxy-3-methyl)-2-pyridyl]methylsufinyl]-5-(1H-pyrrol-1-yl)benzimidazole, polymorphs of 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole or (+)-2-[[(4-methoxy-3-methyl)-2-pyridyl]methylsufinyl]-5-(1H-pyrrol-1-yl)benzimidazole, cocrystals of 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole or (+)-2-[[(4-methoxy-3-methyl)-2-pyridyl]methylsufinyl]-5-(1H-pyrrol-1-yl)benzimidazole, prodrugs of 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole or (+)-2-[[(4-methoxy-3-methyl)-2-pyridyl]methylsufinyl]-5-(1H-pyrrol-1-yl)benzimidazole, polymorphs of 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole or (+)-2-[[(4-methoxy-3-methyl)-2-pyridyl]methylsufinyl]-5-(1H-pyrrol-1-yl)benzimidazole, cocrystals of 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole or (+)-2-[[(4-methoxy-3-methyl)-2-pyridyl]methylsufinyl]-5-(1H-pyrrol-1-yl)benzimidazole or combinations of any of the above.

The terms “administer”, “administering”, “administered” or “administration” refer to any manner of providing a drug to a subject or patient. Routes of administration can be accomplished through any means known by those skilled in the art. Such means include, but are not limited to, oral, buccal, intravenous, subcutaneous, intramuscular, by inhalation and the like.

As used herein, the term “bioavailability” refers to the rate, extent, and duration with which an active ingredient or drug enters and remains in the general circulation, thereby permitting access to the site of action. Higher bioavailability may be achieved, for example, by increasing the active ingredient or drug's duration of action. Methods to determine the bioavailability of active ingredients or drugs are well known to those of ordinary skill in the art.

As used herein, the term “chronic cough” refers to a cough that last for a period of at least one (1) week, preferably at least two (2) weeks and most preferably at least three (3) weeks. Methods of treating chronic cough using PPIs are disclosed in Chung, Clinc. Exp. Allergy, 35:245-246 (2005).

The term “dosage form” refers to any solid object, semi-solid, or liquid pharmaceutical composition designed to contain a specific pre-determined amount (i.e. dose) of a certain active ingredient. Suitable dosage forms may be pharmaceutical drug delivery systems, including those for oral administration, buccal administration, rectal administration, topical or mucosal delivery or subcutaneous implants, or other implanted drug delivery systems and the like. Preferably, the dosage form of the pharmaceutical composition of the present invention is considered to be solid; however, they may contain liquid or semi-solid components. More preferably, the dosage form is an orally administered system for delivering an active ingredient to a patient.

By an “effective amount” or a “therapeutically effective amount” of an active ingredient is meant a nontoxic but sufficient amount of the active ingredient to provide the desired effect. The amount of active ingredient that is “effective” will vary from subject to subject, depending on the age and general condition of the individual, the particular active ingredient or active ingredient, and the like. Thus, it is not always possible to specify an exact “effective amount.” However, an appropriate “effective amount” in any individual case may be determined by one of ordinary skill in the art using routine experimentation.

As used herein, the term “gastrointestinal disorder” refers to any disease or disorder of the upper and lower gastrointestinal tract of a patient including, for example, heartburn, inflammatory bowel disease, Crohn's disease, irritable bowel syndrome, ulcerative colitis, peptic ulcers, stress ulcers, bleeding peptic ulcers, duodenal ulcers, infectious enteritis, colitis, diverticulitis, gastric hyperacidity, dyspepsia, gastroparesis, Zollinger-Ellison syndrome, gastroesophageal reflux disease (“GERD”) (i.e., acid reflux), including, but not limited to, symptomatic GERD and asymptomatic GERD, Helicobacter pylori associated-diseases, hypersecretory states associated with systemic mastocytosis or basophilic leukemia and hyperhistaminemia that result, for example, from neurosurgery, head injury, severe body trauma or burns.

As used herein, the term “lower gastrointestinal tract” refers to the ileum, the colon, the cecum and the rectum.

The term “patient” refers to an animal, preferably a mammal, including a human or non-human. The terms patient and subject may be used interchangeably herein.

By “pharmaceutically acceptable,” such as in the recitation of a “pharmaceutically acceptable excipient,” or a “pharmaceutically acceptable additive,” is meant a material that is not biologically active or otherwise undesirable, i.e., the material may be incorporated into a pharmaceutical composition administered to a patient without causing any undesirable biological effects.

As used herein, the term “stabilizer” refers to any chemical, compound or material that minimizes the degradation of the active ingredient or drug by the acidic environment of the stomach. Examples of stabilizers include, but are not limited to, aluminum salts, bicarbonate salts of aluminum, Group IA metals or Group IIA metal salts (such as, but not limited to, sodium salts, calcium salts, magnesium salts, etc.), bicarbonate salts of Group IA or Group IIA salts (such as a bicarbonate salt of sodium, a bicarbonate salt of magnesium, a bicarbonate salt of calcium), polymers, sodium alginate, sterols, fatty alcohols and combinations thereof.

Examples of polymers that can be used as stabilizers include, but are not limited to, semipermeable homopolymers, semipermeable copolymers, and the like. Preferably, the polymers cellulose esters, cellulose ethers and cellulose ester-ethers. The cellulosic polymers have a degree of substitution (“DS”) of their anhydroglucose unit from greater than 0 up to 3, inclusive. Degree of substitution means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like.

Examples of semipermeable polymers include a member selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%, cellulose diacetate having a DS of 1 to 2 and an acetyl content of 21 to 35%; cellulose triacetate having a DS of 2 to 3 and an acetyl content of 34 to 44.8%, and the like. More specific cellulosic polymers include cellulose propionate having a DS of 1.8 and a propionyl content of 38.5%, cellulose acetate propionate having an acetyl content of 1.5 to 7% and an acetyl content of 39 to 42%, cellulose acetate propionate having an acetyl content of 2.5 to 3%, an average propionyl content of 39.2 to 45%, and a hydroxyl content of 2.8 to 5.4%, cellulose acetate butyrate having a DS of 1.8, an acetyl content of 13 to 15%, and a butyryl content of 34 to 39%, cellulose acetate butyrate having an acetyl content of 2 to 29%, a butyryl content of 17 to 53%, and a hydroxyl content of 0.5 to 4.7%, cellulose triacylates having a DS of 2.6 to 3, such as cellulose trivalerate, cellulose trilamate, cellulose tripalmitate, cellulose trioctanote and cellulose tripropionate, cellulose diesters having a DS of 2.2 to 2.6, such as cellulose disuccinate, cellulose dipalmitate, cellulose dioctanoate, cellulose dicarpylate, and the like; and mixed cellulose esters, such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Semi-permeable polymers comprise cellulose acetaldehyde dimethyl acetate, cellulose acetate ethylcarbamate, cellulose acetate methyl carbamate, cellulose dimethylaminoacetate, semipermeable polyamide, semipermeable polyurethanes; semipermeable sulfonated polystyrenes, cross-linked selectively semipermeable polymers formed by the coprecipitation of an anion and a cation, as disclosed in U.S. Pat. Nos. 3,173,876, 3,276,586, 3,541,005, 3,541,006 and 3,546,142, semipermeable polymers, as disclosed by Loeb, et al. in U.S. Pat. No. 3,133,132, semipermeable polystyrene derivatives, semipermeable poly(sodium styrenesulfonate), semipermeable poly(vinylbenzyltrimethylammonium chloride); and semipermeable polymers exhibiting a fluid permeability of 10⁻⁵ to 10⁻² (cc. mil/cm hr.atm), expressed as per atmosphere of hydrostatic or osmotic pressure differences across a semipermeable wall. The polymers known to those skilled in the art are described in U.S. Pat. Nos. 3,845,770, 3,916,899 and 4,160,020; and in Handbook of Common Polymers, Scott and Roff (1971) CRC Press, Cleveland, Ohio.

Examples of sterols that can be used as stabilizers include, but are not limited to, phytosterols (such as ergosterols, stigmasterol, sitosterol, brassicasterol and campesterol), zoosterols (such as cholesterol and lanosterol) or combinations thereof.

The fatty alcohols that can be used as stabilizers can be linear, saturated or unsaturated primary alcohols having 10-30 carbon atoms. Examples of fatty alcohols that can be used include, but are not limited to, cetyl alcohol, myristyl alcohol or stearyl alcohol.

The terms “treating” and “treatment” refer to a reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the term “ulcers” refers to lesions of the upper gastrointestinal tract lining that are characterized by a loss of tissue. Such ulcers include, but are not limited to, gastric ulcers, duodenal ulcers and gastritis.

As used herein, the term “upper gastrointestinal tract” refers to the esophagus, the stomach, the duodenum and the jejunum.

The fasting pH of the stomach varies between a pH of 2 to 6 (a pH of less than 7 is considered to be an acidic pH). The pH of the small intestine is more alkaline than the pH of the stomach and increases from the duodenum to the ileum. The active ingredient of the present invention, like other PPI's known in the art, is acid labile. It rapidly degrades at an acidic pH to an inactive compound. When a tablet or capsule dissolves in the stomach, this tablet or capsule is thoroughly mixed with the gastric contents of the stomach. Upon transferring from the stomach to the duodenum, the gastric contents are slowly neutralized by bicarbonate present in duodenum. Thus, the pH increases as the gastric contents transition through the small intestine.

The exact location of drug absorption, whether in the stomach, small intestine or throughout the gastrointestinal tract, is uncertain. The inventors of the present invention discovered that the active ingredient exhibits site-specific absorption in the upper part of the small intestine (See Example 4). Specifically, the absorption of the active ingredient is significantly higher in the upper part of the small intestine, namely in the area of the duodenum, in the area of the upper jujenum or a combination of the areas of the duodenum and upper jujenum, where the pH is more acidic.

The present invention relates to pharmaceutical compositions comprising solid particles of an active ingredient, wherein the solid particles have a mean particle size from about 0.1 microns to about 100 microns. Preferably, the pharmaceutical compositions of the present invention comprise solid particles of an active ingredient having a mean particle size from about 0.5 microns to about 75 microns. More preferably, the pharmaceutical compositions of the present invention comprise solid particles of an active ingredient having a mean particle size from about 0.75 microns to about 65 microns. Even more preferably, the pharmaceutical compositions of the present invention comprise solid particles of an active ingredient having a mean particle size from about 1 micron to about 50 microns. The present invention also contemplates pharmaceutical compositions comprising solid particles having a particle size less than about 50 microns. More preferably, the pharmaceutical compositions of the present invention comprise solid particles of an active ingredient having a mean particle size of less than about 45 microns. And even more preferably, the pharmaceutical compositions of the present invention comprise solid particles of an active ingredient having a mean particle size of less than about 40 microns.

The mean particle size of solid particles of the active ingredient contained in the pharmaceutical compositions described herein (namely, between 0.1 micron and 100 microns) is necessary to insure the maximum amount of drug absorption (thus leading to greater bioavailability of the active ingredient) with the least amount of degradation after ingestion of the pharmaceutical compositions described herein. Thereupon, solid particles of active ingredient having the mean particle size described herein provide a large surface area with less degradation and thus higher absorption potential than solid particles of active ingredient having mean particle sizes larger than the particle sizes described herein (namely, solid particles of the active ingredient having a particle size greater than 100 microns). In contrast, solid particles of active ingredient having a mean particle size greater than 100 microns provide less degradation but also less absorption.

It is contemplated that the pharmaceutical compositions of the present invention may contain a small amount of solid particles of the active ingredient that have a mean particle size greater than about 100 microns. However, it is preferred that the pharmaceutical compositions of the present invention do not contain more than 10% of solid particles of the active ingredient having a mean particle size larger than 100 microns Most preferably, the pharmaceutical compositions of the present invention do not contain more than 5% of solid particles of the active ingredient having a mean particle size larger than 100 microns.

Solid particles of the active ingredient that have a mean particle size between 0.1 micron and 100 microns can be made using routine techniques known in the art. For example, such particles can be made by micronizing raw material of the active ingredient. Any technique for micronizing known in the art can be used provided that said technique produces particles between 0.1 microns and 100 microns. Examples of such techniques that can be used include, but are not limited to, wet milling, high pressure homogenization, emulsification and precipitation, precipitation with a compressed fluid anti-solvent (such as super critical CO₂ mixed with an organic solvent containing the active ingredient), spray freezing into a liquid (namely, where a solution or suspension containing the active ingredient is atomized into a cryogenic liquid to produce frozen nanoparticles followed by freeze-drying); rapid expansion from a liquefied-gas solution (such as where the active ingredient and a surfactant are dissolved in a super critical fluid followed by rapid expansion), evaporative precipitation into an aqueous solution (such as where a solution containing the active ingredient is placed under pressure and heated to a temperature above the boiling point of the solvent and then atomized into a heated aqueous solution containing a stabilizing suspension), grinding, milling, ball milling and air jet micronization.

Methods for determining the particle size of solid particles of an active ingredient are well known to those skilled in the art. For example, a Sympatech HELOS particle size system (commercially available from Sympatech GmBH, Clausthal-Zellerfeld, Germany) can be used to determine the particle size of the solid particles of the pharmaceutical composition of the present invention. The Sympatech HELOS particle size system operates using low-angle laser light scattering (“LALLS”) that is analyzed by Fraunhofer diffraction theory. The Fraunhofer diffraction theory is described Frank L. Pedrotti and Leno S. Pedrotti, Introduction to Optics, 2^{nd} Edition (Nov. 16, 2002). Other techniques that are known in the art that can be used to determine the particle size of solid particles of an active ingredient include, but are not limited to, electrozone particle counter, low angle laser light scattering, capillary hydrodynamic fractionation, optical particle counter, competitive disc centrifuge, sedimentation field flow fractionation and CPS disc centrifuge.

The “mean particle size” of an active ingredient comprising solid particles of an active ingredient comprising 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof can be determined using routine techniques known in the art. For example, a representative sample of solid particles can be obtained from a pharmaceutical composition (such as a tablet or capsule) and the size of the solid particles contained in the representative sample determined using routine techniques known in the art, including, but not limited to, electrozone particle counter, low angle laser light scattering, capillary hydrodynamic fractionation, optical particle counter, competitive disc centrifuge, sedimentation field flow fractionation and CPS disc centrifuge. The “mean” value of the size of the solid particles contained in the sample can then be calculated using the particle size for each of the solid particles contained in the sample and determined using the techniques described herein. This mean would represent the “mean particle size” of the solid particles of the active ingredient contained within the pharmaceutical composition.

The pharmaceutical compositions of the present invention are particularly desirable for use in treating gastrointestinal disorders, particularly, but not limited to, symptomatic GERD, dyspepsia and heart bum, where providing pain relief as quickly as possible after administration of the pharmaceutical composition is desired. Moreover, because the pharmaceutical compositions of the present invention exhibit higher bioavailability, this may allow a reduction in the dose that would need to be administered to a patient in need of treatment thereof.

The pharmaceutical compositions of the present invention comprising solid particles of an active ingredient comprising 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof (hereinafter collectively referred to as “Ilaprazole”) and having mean particles sizes within the ranges described herein provide a number of benefits. Specifically, these pharmaceutical compositions exhibit higher (or greater) bioavailability when administered to a patient in need of treatment thereof when compared to pharmaceutical compositions that contain solid particles of an active ingredient wherein 40% or more of said solid particles have a mean particle size greater than 100 microns (with 25% of the particles having a mean particle size greater than 200 microns). Additionally, these pharmaceutical compositions exhibit a faster onset of action compared to pharmaceutical compositions that contain solid particles of an active ingredient where 40% or more of said solid particles have a mean particle size greater than 100 microns (with 25% of the particles having a mean particle size greater than 200 microns). The finding that these pharmaceutical compositions exhibit a higher bioavailability was unexpected. Specifically, it is known in the art that with any active ingredient that there has to be a balance between the in vitro stability of the active ingredient and the in vivo stability and bioavailability of the active ingredient. If the degradation rate of the active ingredient in vivo is greater than the absorption rate of the active ingredient in vivo, then the bioavailability of the active ingredient will decline and visa versa. Given the rapid rate at which the solid particles of the active ingredient of these pharmaceutical compositions degrade in vivo, the higher (greater) bioavailability of these compositions in the upper part of the small intestine was unexpected. In fact, the inventors expected that the size of the solid particles of the active ingredient of these pharmaceutical compositions coupled with the location of the absorption of the active ingredient (in the upper part of the small intestine) would have resulted in more degradation of the active ingredient and thus reduced bioavailability.

Additionally, the pharmaceutical compositions of the present invention comprising solid particles of an active ingredient comprising 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof (hereinafter collectively referred to as “Ilaprazole”) and having mean particles sizes within the ranges described herein have an in vitro dissolution profile in which at least 70%, at least 75%, at least 80%, at least 85% and at least 90% of the active ingredient thereof is dissolved (or released from the composition) within 20 minutes. In comparison, less than at least 70% of the active ingredient in a pharmaceutical composition that contain solid particles of an active ingredient wherein 40% or more of said solid particles have a mean particle size greater than 100 microns (with 25% of the particles having a mean particle size greater than 200 microns) is dissolved within about 20 minutes. The dissolution profile may be measured using the following dissolution test: dissolution test Apparatus 1 (basket method) in 500 mL of a pH 7.5 buffer with about 0.5 sodium lauryl sulfate as the dissolution medium, at about 100 rpm and at a temperature of about 37° C.±0.5° C.

The benefits of the pharmaceutical compositions described herein are not limited to a particular type of dosage form having a specific mechanism of active ingredient or drug release. The benefits of the pharmaceutical compositions of the present invention can be obtained with any dosage form that is suitable for releasing an active ingredient such as, for example, a continuous release of the drug. In view of the discovery regarding the particle size of the solid particles of the active ingredient, the method of delivery of the active ingredient is a matter of choice for those skilled in the art.

Many types of continuous drug release dosage forms are known in the art. For example, controlled or extended release, and pulsed release dosage forms are known. Any type of continuous drug release dosage form can be used in the present invention, including matrix systems, osmotic pumps, and membrane controlled systems (also referred to as reservoir systems). Each of these systems is described in greater detail below. A detailed discussion of such dosage forms may also be found in: (i) Handbook of pharmaceutical controlled release technology, ed. D. L. Wise, Marcel Dekker, Inc. New York, N.Y. (2000), and (ii) and Treatise on controlled drug delivery, fundamentals, optimization, and applications, ed. A. Kydonieus, Marcel Dekker, Inc. New York, N.Y. (1992).

Matrix systems are well known in the art. In a matrix system, the drug is homogenously dispersed in a polymer and optionally, conventional excipients. This so-called admixture is typically compressed under pressure to produce a tablet. Drug is released from this tablet by diffusion and erosion. Matrix systems typically employ a pharmaceutically acceptable polymer such as a water-soluble hydrophilic polymer, or a water insoluble hydrophobic polymer (including waxes). One skilled in the art would readily be able to determine the type of pharmaceutically acceptable polymer to be used using routine techniques to those known in the art.

The pharmaceutical compositions of the present invention also typically include pharmaceutically acceptable excipients. As is well known to those skilled in the art, pharmaceutical excipients are routinely incorporated into solid dosage forms. This typically is done to ease the manufacturing process as well as to improve the performance of the pharmaceutical composition. Common excipients include, but are not limited to, diluents or bulking agents, lubricants, binders, etc.

Diluents, or fillers, can be added to, for example, increase the mass of an individual dose to a size suitable for tablet compression. Suitable diluents include, but are not limited to, powdered sugar, calcium phosphate, calcium sulfate, microcrystalline cellulose, lactose, mannitol, kaolin, sodium chloride, dry starch, xylitol and sorbitol.

Lubricants can be incorporated into a pharmaceutical composition for a variety of reasons. They reduce friction between the granulation and die wall during compression and ejection. This prevents, for example, a granulate from sticking to the tablet punches, and facilitates its ejection from the tablet punches. Examples of suitable lubricants include, but are not limited to, talc, stearic acid, vegetable oil, calcium stearate, zinc stearate, magnesium stearate, solid polyethylene glycols, sodium stearyl fumarate, silica gel, glyceryl behenate mixtures thereof and other substances with lubricating properties.

Glidant's can also be incorporated into a pharmaceutical composition, typically for purposes of improving the flow characteristics of the granulation. Examples of suitable glidant's include, but are not limited to, talc, silicon dioxide, and cornstarch.

Binders also may be incorporated into the pharmaceutical composition of the present invention. Binders are typically utilized if the manufacture of the dosage form employs a granulation step. Examples of suitable binders include povidone (such as polyvinylpyrrolidone), sugars (such as sucrose), xanthan gum, cellulose gums such as carboxymethylcellulose, methyl cellulose, hypromellose, microcrystalline cellulose, hydroxycellulose, hydroxypropylcellulose, mallodextrin gelatin, starch, pregelatinized starch, and other pharmaceutically acceptable substances with cohesive properties.

Other excipients that may be incorporated into the pharmaceutical composition include absorption accelerators, absorbents, effervescent agents, emulsifers, disintegrating agents, humectants, preservatives, solution retarders, solubility enhancing agents, buffers, surfactants, suspending agents, sweeteners, wetting agents or any other pharmaceutically acceptable excipient commonly used in the pharmaceutical industry.

Examples of “absorption accelerators” that can be used in the present invention include, but are not limited to, quaternary ammonium compounds. Examples of “absorbents” that can be used in the present invention include, but are not limited to, kaolin and bentonite. Examples of “effervescent agents” that can be used in the present invention are effervescent couples such as, but not limited to, an organic acid and a carbonate or bicarbonate. Suitable organic acids include, but are not limited to, citric, tartaric, malic, fumaric, adipic, succinic, and alginic acids and anhydrides and acid salts. Suitable carbonates and bicarbonates include, but are not limited to, sodium carbonate, sodium bicarbonate, potassium carbonate, potassium bicarbonate, magnesium carbonate, sodium glycine carbonate, L-lysine carbonate and arginine carbonate. Examples of “emulsifiers” that can be used in the present invention include, but are not limited to, ethyl alcohol, isopropyl alcohol, ethyl carbonate, ethyl acetate, benzyl alcohol, benzyl benzoate, propylene glycol, 1,3-butylene glycol, dimethylformamide, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like. Examples of “disintegrating agents” that can be used in the present invention include, but are not limited to, lightly cross-linked polyvinyl pyrrolidone, corn starch, potato starch, maize starch and modified starches, agar-agar, calcium carbonate, sodium carbonate, alginic acids, cross carmellose sodium, cross povidone, sodium starch glycolate and mixtures thereof. Examples of “humectants” that can be used in the present invention, include, but are not limited to, glycerol. Examples of “preservatives” that can be used in the present invention include, but are not limited to, potassium sorbate, methylparaben, propylparaben, benzoic acid and its salts, other esters of parahydroxybenzoic acid such as butylparaben, alcohols such as ethyl or benzyl alcohol, phenolic compounds such as phenol or quaternary compounds such as benzalkonium chloride. Examples of “solution retarders” that can be used include in the present invention include, but are not limited to, paraffin. Examples of “solubility enhancing agents” that can be used in the present invention include, but are not limited to, co-solvents such as ethanol or propylene glycol, surfactants and polymeric substances such as polysorbates, polyalkylene glycols, poloxamers or polyvinylpyrrolidone, and oily fatty acids and their mono- or diglyceryl esters such as linoleic acid or glyceryl monolaurate. Examples of suitable “buffers” that can be used in the present invention include, but are not limited to, phosphate, acetate, citrate, succinate and histidine buffers. The term “surfactant” is used in its conventional sense in this invention. Any surfactant is suitable, whether it is amphoteric, non-ionic, cationic or anionic. Examples of suitable surfactants include, but are not limited to, sodium lauryl sulfate, monooleate monolaurate, monopalmitate, monstearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, polyoxyethylene sorbitan fatty acid esters (e.g., the commercially available Tween®s, such as, Tween® 20 and Tween® 80 (ICI Speciality Chemicals)), polyethylene glycols (e.g., Carbowaxs 3550® and 934® (Union Carbide)), poloxamers (e.g., Pluronics F68® and F108®, which are block copolymers of ethylene oxide and propylene oxide); polyoxyethylene castor oil derivatives or mixtures thereof. Examples of “suspending agents” that can be used include in the present invention include, but are not limited to, carboxymethylcelluose, veegum, tragacanth, bentonite, methylcellulose and polyethylene glycols. Examples of “sweeteners” that can be used in the present invention include, but are not limited to, any natural or artificial sweetener such as, but not limited to, sucrose, xylitol, sodium saccharin, cyclamate, aspartame and acsulfame. Examples of flavoring agents are Magnasweet®, bubble gum flavor, fruit flavors and the like. Examples of “wetting agents” that can be used in the present invention include, but are not limited to, ammonium lauryl sulfate and sodium lauryl sulfate.

The amount of excipients used in the pharmaceutical composition will correspond to that typically used in a matrix system. The total amount of excipients, fillers and the like typically will vary from about 10% to about 80% by weight of the pharmaceutical composition.

Matrix dosage forms of pharmaceutical compositions are generally prepared using standard techniques well known in the art. Typically, they are prepared by dry blending the polymer, filler, drug, and other excipients followed by granulating the mixture using an alcohol until proper granulation is obtained. The granulation is done by methods known in the art. The wet granules are dried in a fluid bed dryer, sifted and ground to appropriate size. Lubricating agents are mixed with the dried granulation to obtain the final pharmaceutical composition.

In an osmotic pump system, a tablet core is encased by a semipermeable membrane having at least one orifice. The semipermeable membrane is permeable to water, but impermeable to the drug. When the system is exposed to body fluids, water will penetrate through the semipermeable membrane into the tablet core containing osmotic excipients and the active drug. Osmotic pressure increases within the pharmaceutical composition and drug is released through the orifice in an attempt to equalize pressure.

In more complex pumps, the tablet core contains multiple internal compartments. For example, the first compartment may contain the drug and the second compartment may contain a polymer that swells on contact with fluid. After ingestion, this polymer swells into the drug containing compartment at a predetermined rate and forces drug from the pharmaceutical composition at that rate. Such pharmaceutical compositions are often used when are zero order release profile is desired.

Osmotic pumps are well known in the art and have been described in the literature. U.S. Pat. Nos. 4,088,864, 4,200,098, and 5,573,776, all of which are hereby incorporated by reference, describe osmotic pumps and methods for their manufacture. Osmotic pumps containing compounds, such as omeprazole, have been described in U.S. Pat. No. 5,178,867, the contents of which are hereby incorporated by reference.

As a general guideline, osmotic pumps are typically formed by compressing a tablet of an osmotically active drug (or an osmotically inactive drug in combination with an osmotically active agent or osmagent) and then coating the tablet with a semipermeable membrane that is permeable to an exterior aqueous-based fluid but impermeable to the passage of drug and/or osmagent. One or more delivery orifices may be drilled through the semipermeable membrane wall. Alternatively, orifice(s) through the wall may be formed in situ by incorporating leachable pore forming materials in the wall. In operation, the exterior aqueous based fluid is imbibed through the semipermeable membrane wall and contacts the drug and/or salt to form a solution or suspension of the drug. The drug solution or suspension is then pumped out through the orifice as fresh fluid is imbibed through the semipermeable membrane.

As previously mentioned, osmotic pumps may contain multiple distinct compartments. The first compartment may contain the drug as described above, and the second compartment may contain an expandable driving member consisting of a layer of a swellable hydrophilic polymer, which operates to diminish the volume occupied by the drug, thereby delivering the drug from the device at a controlled rate over an extended period of time. Alternatively, the compartments may contain separate doses of the drug.

Typical materials for the semipermeable membrane include semipermeable polymers known to the art as osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, cross-linked selectively semipermeable polymers formed by the coprecipitation of a polyanion and a polycation as disclosed in U.S. Pat. Nos. 3,173,876; 3,276,586, 3,541,005, 3,541,006, and 3,546,142, semipermeable polymers as disclosed by Loeb and Sourirajan in U.S. Pat. No. 3,133,132, lightly cross-linked polystyrene derivatives, cross-linked poly(sodium styrene sulfonate), poly(vinylbenzyltrimethyl ammonium chloride), cellulose acetate having a degree of substitution up to 1 and an acetyl content up to 50%, cellulose diacetate having a degree of substitution of 1 to 2 and an acetyl content of 21 to 35%, cellulose triacetate having a degree of substitution of 2 to 3 and an acetyl content of 35 to 44.8%, as disclosed in U.S. Pat. No. 4,160,020.

The osmotic agent present in the pump, which may be used when the drug itself is not sufficiently osmotically active, are osmotically effective compounds soluble in the fluid that enters the pump, and exhibits an osmotic pressure gradient across the semipermeable wall against the exterior fluid. Osmotically effective osmagents useful for the present purpose include magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, mixtures thereof, and the like. The osmagent is usually present in an excess amount, and it can be in any physical form, such as particle, powder, granule, and the like. The osmotic pressure in atmospheres of the osmagents suitable for the invention will be greater than zero and generally up to about 500 atm, or higher.

The expandable driving member typically is a swellable, hydrophilic polymer which interacts with water and aqueous biological fluids and swells or expands to an equilibrium state. The polymers exhibit the ability to swell in water and retain a significant portion of the imbibed water within the polymer structure. The polymers swell or expand to a very high degree, usually exhibiting a 2 to 50 fold volume increase. The polymers can be cross-linked or may not be cross-linked. The swellable, hydrophilic polymers can be lightly cross-linked, such cross-links being formed by covalent ionic bonds or hydrogen bonds. The polymers can be of plant, animal or synthetic origin. Hydrophilic polymers that can be used in for the present invention include poly(hydroxy alkyl methacrylate) having a molecular weight from 30,000 to 5,000,000; kappa carrageenan, polyvinylpyrrolidone having molecular weight of from 10,000 to 360,000; anionic and cationic hydrogels; polyelectrolyte complexes; poly(vinyl alcohol) having a low acetate residual, cross-linked with glyoxal, formaldehyde, or glutaraldehyde and having a degree of polymerization from 200 to 30,000; a mixture of methyl cellulose; cross-linked agar and carboxymethyl cellulose; a water insoluble, water swellable copolymer produced by forming a dispersion of finely divided copolymer of maleic anhydride with styrene, ethylene, propylene, butylene or isobutylene cross-linked with from 0.001 to about 0.5 moles of saturated cross-linking agent per mole of maleic anhydride in copolymer; water swellable polymers of N-vinyl lactams, and the like.

The term “orifice” as used herein refers to means and methods suitable for releasing the drug from an osmotic system. The expression includes one or more apertures or orifices which have been bored through the semipermeable membrane by mechanical procedures. Alternatively, it may be formed by incorporating an erodible element, such as a gelatin plug, in the semipermeable membrane. In cases where the semipermeable membrane is sufficiently permeable to the passage of drug, the pores in the membrane may be sufficient to release the active ingredient in amounts sufficient to meet the plasma threshold. In such cases, the term “passageway” refers to the pores within the membrane wall even though no bore or other orifice has been drilled there through. A detailed description of osmotic passageways and the maximum and minimum dimensions for a passageway are disclosed in U.S. Pat. Nos. 3,845,770 and 3,916,899, the disclosures of which are incorporated herein by reference.

Osmotic pumps can be manufactured by standard techniques. For example, in one embodiment, the drug and other ingredients that may be housed in one area of the compartment adjacent to the passageway, are pressed into a solid possessing dimension that corresponds to the internal dimensions of the area of the compartment the drug will occupy, or the drug and other ingredients and a solvent are mixed into a solid or semisolid form by conventional methods such as ballmilling, calendaring, stirring or rollmilling, and then pressed into a preselected shape. Next, a layer of a hydrophilic polymer is placed in contact with the layer of drug in a like manner, and the two layers surrounded with a semipermeable wall. The layering of drug formulation and hydrophilic polymer can be fabricated by conventional two-layer press techniques. The wall can be applied by molding, spraying or dipping the pressed shapes into a wall forming material. Another and presently preferred technique that can be use for applying the wall is the air suspension procedure. This procedure consists of suspending and tumbling the pressed agent and dry hydrophilic polymer in a current of air and a wall forming composition until the wall is applied to the agent-hydrophilic polymer composite. The air suspension procedure is described in U.S. Pat. No. 2,799,241; J Am. Pharm. Assoc., 48:451-459 (1979). Other standard manufacturing procedures are described in Modern Plastics Encyclopedia, Vol. 46, pp. 62-70 (1969); and in Pharmaceutical Sciences, by Remington, Fourteenth Edition, pp. 1626-1678 (1970), published by Mack Publishing Company, Easton, Pa.

Reservoir systems also are well known in the art. This technology is also commonly referred to as microencapsulation, bead technology, or coated tablets. Particles of the drug are encapsulated with pharmaceutically acceptable polymer. This polymer, and its relative quantity, offers a predetermined resistance to drug diffusion from the reservoir to the gastrointestinal tract. Thus drug is gradually released from the beads into the gastrointestinal tract and provides the desired sustained release of the compound.

These dosage forms of pharmaceutical compositions are well known in the art. U.S. Pat. Nos. 5,286,497 and 5,737,320, both of which are hereby incorporated by reference, describe such dosage forms and their methods of production. U.S. Pat. Nos. 5,354,556, 4,952,402, and 4,940,588, all of which are hereby incorporated by reference, specifically discuss using such technology to produce sustained release pharmaceutical compositions. As further guidance, however, a pellet is formed with a core of a drug, optionally in association with conventional excipients. This core is then coated with one, or more, pharmaceutically acceptable polymers. Often, the coating polymer is an admixture of a major proportion of a pharmaceutically acceptable water insoluble polymer and a minor proportion of a pharmaceutically acceptable water soluble polymer.

The central core may be prepared by a number of techniques known in the art. Typically the drug is bound to an inert carrier with a conventional binding agent. The inert carrier is typically a starch or sugar sphere. Before the drug is bound to the inert carrier, it is typically blended with conventional excipients to expedite its handling and to improve the properties of the final dosage form of the pharmaceutical composition. These excipients are identical to those described above for the matrix systems. The quantity of these excipients can vary widely, but will be used in conventional amounts. The central core is then produced by utilizing a binding agent to attach the powdered drug blend to the solid carrier. This can be accomplished by means known in the art for producing pharmaceutical beads. Suitable means include utilization of a conventional coating pan, an automatic coating machine, or a rotogranulator. The production of these central cores is described in more detail in Pharmaceutical Pelletization Technology, ed. I. GhebreSellassie, Marcel Dekker, Inc. New York, N.Y. (1989).

The second major component of a reservoir system is the polymeric coating. As noted above, the polymeric coating is responsible for giving the beads their release characteristics. The polymeric coating may be applied to the central core using methods and techniques known in the art. Examples of suitable coating devices include fluid bed coaters and pan coaters. The application techniques are described in more detail in: i) Aqueous polymeric coatings for pharmaceutical pharmaceutical compositions, ed. J. W. McGinity, Marcel Dekker, Inc. New York, N.Y. (1997); and ii) Pharmaceutical compositions: Tablets Vol. 3. ed. H. A. Lieberman, L. Lachman and J. B. Schwartz, Marcel Dekker, Inc. New York, N.Y. pp. 77-287, (1990).

Examples of suitable polymers include ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride) or polyurethane or mixtures thereof.

Once the beads have been prepared, they may be filled into capsules as is known in the art. Alternately, they may be pressed into tablets using techniques conventional in the art.

Pulsed release systems, the other broad category of modified release dosage forms of pharmaceutical compositions, are also well known in the art. Pulsed release systems generally involve a first drug release and a second drug release separated by a predetermined period of time or site of release. Pulsed release systems also may include a combination of immediate release and extended release. Multiple formulation configurations are suitable for pulsed release dosage forms of pharmaceutical compositions.

For example, osmotic pumps also are suitable for purposes of pulsatile drug release and have been described in U.S. Pat. Nos. 5,017,381 and 5,011,692, both of which are herein incorporated by reference. Generally, the osmotic pump containing the drug is formed and then overcoated with a layer of a drug to provide for two releases of the drug, one from the coating layer and another from the osmotic pump.

Particle or granule systems have also been proposed for purposes of providing a pulsed release of drug. Systems for the pulsed release of a drug typically use distinct populations of drug containing particles to achieve a pulsed release. The populations employ different coating polymers, such as those mentioned above, to release the drug at different points in time or location. For example, polymers having different dissolution pHs are commonly used for this purpose. Hence, one population of granules can be coated with a polymer that begins dissolving at a pH of 6 and another population of granules can be coated with a polymer that begins dissolving at a pH of 6.5 to achieve a pulsed release. In this manner, the first population of granules would release the drug in the upper small intestine while the second population of the granules would release the drug further down stream and therefore at a later time.

It will be understood, of course, that the pharmaceutical compositions of the present invention may employ an enteric coating or buffering systems such as those described in U.S. Pat. Nos. 6,849,346, 5,026,560, 5,045,321, 4,786,505 and 6,849,346 (all of which are herein incorporated by reference) for purposes of protecting the active ingredient. Examples of an enteric coating that can be used include, but are not limited to, cellulose acetate phthalate, hydroxypropyl methylcellulose phthalate, hydroxypropyl methylcellulose acetate succinate, polyvinyl acetate phthalate, anionic polymers of methacrylic acid and methacrylates (such as, for example, EUDRAGIT® S 12.5, S 12.5 P, S 100, etc.), cellulose acetate trimellitate, shellac and combinations thereof (See, Raymond Roe, Paul Sheskey and Sian Owen, Handbook of Pharmaceutical Excipients, 5^{th} Edition (Dec. 14, 2005), APhA Publications). One skilled in the art could readily determine the type of enteric coating to be used. If an enteric coating is used, a coating between the active ingredient and enteric coating can also be used (such a coating is frequently referred to as a “subcoating”). Any film forming polymer can be used as a subcoating. For example, polymers such as polyvinyl alcohol, hydroxypropyl cellulose, hypromellose can be used.

It will be understood, of course, that the pharmaceutical compositions of the present invention may be in the form of a controlled release preparation as described in WO 2004/035020, the contents of which are herein incorporated by reference. WO 2004/035020 describes a preparation containing a gel-forming polymer and an active ingredient wherein the release of the active ingredient is controlled.

The pharmaceutical compositions of the present invention can be administered orally in the form of tablets, pills, or granules may be loose filled into capsules. The tablets can be prepared by techniques known in the art and contain a therapeutically effective amounts of the active ingredient and such excipients as are necessary to form the tablet by such techniques. Tablets and pills can additionally be prepared with enteric coatings and buffering systems such as those described above to protect the active ingredient. The coating may be colored with a pharmaceutically accepted dye. The amount of dye and other excipients in the coating liquid may vary and will not impact the performance of the extended release tablets. The coating liquid generally comprises film forming polymers such as hydroxypropyl cellulose, hypromellose, cellulose esters or ethers (such as cellulose acetate or ethylcellulose), an acrylic polymer or a mixture of polymers. The coating solution is generally an aqueous solution or an organic solvent further comprising propylene glycol, sorbitan monoleate, sorbic acid, fillers such as titanium dioxide, a pharmaceutically acceptable dye.

One skilled in the art, taking into account above teachings will be readily able to formulate pharmaceutical compositions containing the active ingredient having the particle sizes recited herein.

As discussed briefly herein, the pharmaceutical compositions of the present invention can be used to treat a patient suffering from a gastrointestinal disorder and in need of treatment thereof. Such a patient can be treated by administering to said patient a therapeutically effective amount of the pharmaceutical composition of the present invention. Moreover, the pharmaceutical compositions of the present invention can also be used to treat a patient suffering from chronic cough and in need of treatment thereof. Such a patient can be treated by administering to said patient a therapeutically effective amount of the pharmaceutical composition of the present invention.

By way of example and not of limitation, examples of the present invention will now be given.

### EXAMPLE 1

### Particle Size of Ilaprazole

Raw material of ilaprazole was synthesized at Raylo Chemicals Inc., Alberta, Canada and micronized to give Lot A. Lot B was obtained from Il-Yang Pharmaceutical Company, Seoul, South Korea.

The two lots of ilaprazole active ingredient were analyzed using a Sympatec HELOS particle size system with a RODOS dry powder disperser. This instrument operates using low-angle laser light scattering analyzed by Fraunhofer diffraction theory.

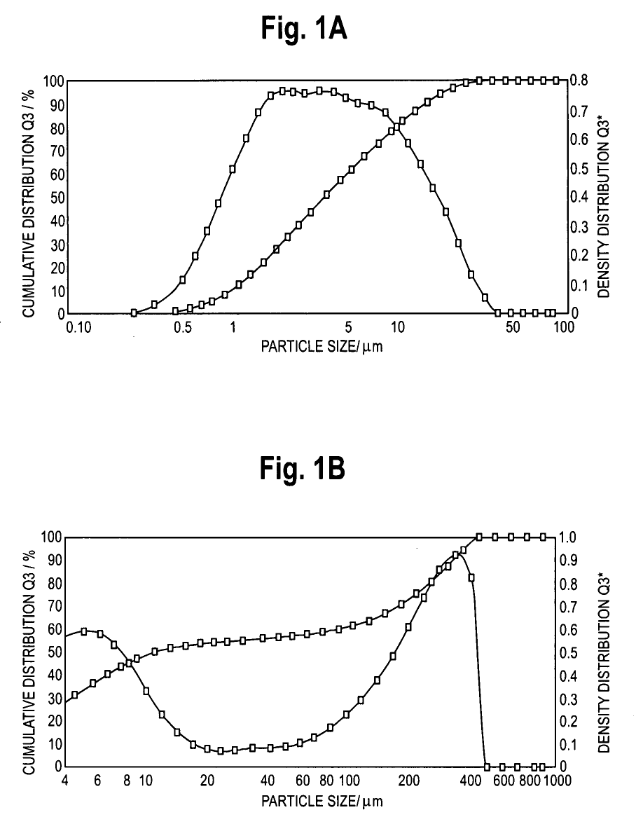

Aliquots of approximately 0.5 g were fed into the instrument from a vibrating tray into a jet of nitrogen gas. After flowing through an interaction tube, an aerosol was formed in the path of a laser beam. The angle and intensity of diffracted light was then measured to determine the particle size distribution. As part of the method development, the particle size distribution was measured as a function of nitrogen gas pressure, and a pressure of 2 bar was determined to adequately break apart loosely adhered particles without significant milling of the sample. The results are shown below in Table 1 and in FIG. 1.

TABLE 1

| | x₁₀ | x₅₀ | x₉₀ |
|---|---|---|---|
| Lot | Mean | Mean | Mean |
| A (Micronized) | 1.01 μm | 3.74 μm | 14.79 μm |
| B | 1.80 μm | 11.41 μm | 333.07 μm |

The particle sizes are listed as x₁₀, x₅₀ and x₉₀, the diameter where 10%, 50% and 90% of the volume of the material is in a particle smaller than the listed size.

### EXAMPLE 2

### Ilaprazole Containing Formulations

The following steps were utilized during the manufacturing of below described formulations A and B:
1. Drug layering
2. Subcoating
3. Enteric coating
4. Capsule filling
   Drug Layering

Ilaprazole (from Lot A in Example 1) was layered (coated) on to either sugar spheres (which is referred to hereinafter as “Formulation A”) or Celphere (which is commercially available from Asahi Kasei, Japan) (which is referred to hereinafter as “Formulation B”) by fluid bed processing (bottom spray with partition). The layering process for both formulations was the same. The layering suspension was prepared as follows. Purified water was weighed into a beaker. Hydroxypropyl cellulose (“HPC-L”) was gradually added (to prevent clumping) with stirring until a solution resulted. Magnesium carbonate and low substituted hydroxypropyl cellulose (“L-HPC”) were homogenously dispersed in this solution by utilizing vigorous stirring. Finally, ilaprazole (Lot A from Example 1) was added slowly to this suspension and homogenously dispersed. The resulting suspension was stirred for 30 minutes and passed through a 20-mesh screen to ensure no aggregates or lumps remained. The composition of layering suspension utilized for Formulations A and B is presented in Table 2 below.

TABLE 2

| Ingredient | Function | % w/w |
|---|---|---|
| Purified Water | Solvent | 72.0 |
| L-HPC | Disintegrant | 2.5 |
| HPC-L | Binder | 5.0 |
| Ilaprazole | Active | 15.5 |
| Magnesium carbonate | Stabilizer | 5.0 |

Sugar spheres (Formulation A) or Celpheres (Formulation B) were coated with the layering suspension by a fluid bed process that utilized bottom spray technique. The fluid bed processor (FluidAir Model 0002, Aurora, Ill.) was preheated for about 5 minutes before placing weighed sugar spheres or Celpheres into the bowl. Fluidization was started and layering suspension was sprayed onto sugar spheres or Celpheres. Layering parameters are summarized in Table 3, below. At the end of the layering run, product temperature was allowed to increase by 3-4° C. to dry the product and the beads were discharged.

TABLE 3

| Process Parameter | Layering Run 1 | Layering Run 2 |
|---|---|---|
| Inlet Air (SCFM) | 8-9 | 9 |
| Inlet Temp (° C.) | 56.7-63.5 | 54.9-61.9 |
| Outlet Temp (° C.) | 30.0-32.3 | 27.6-33.1 |
| Product Temp (° C.) | 36.0-41.5 | 33.0-40.9 |
| Spray Air (PSI) | 20-25 | 25 |
| Pump setting (%) | 10-20 | 12 |
| (rate g/min) | (2.0-3.3) | (3.3) |
| Filter Blow Back (PSI) | 45-65 | 30-65 |

Subcoating

Opadry II Y-30-18037 (Colorcon, Pa.) was dissolved in water to form a 17.5% w/w suspension. Subcoating suspension was prepared in excess 3 times the desired amount and an amount corresponding to a 10% weight gain of solids was applied. To prepare the subcoating suspension, purified water was weighed in a suitable container and stirred vigorously to form a vortex. Opadry II powder was added slowly to prevent formation of lumps. After all the powder has been added, the suspension was stirred for about 20 minutes. Processing parameters were similar to those used in the layering experiments. Higher product temperatures (40-43° C.) were obtained due to lower spray rates used. There was no agglomeration observed during the sub-coating process. Approximately 162 g of subcoated beads were discharged at the end of the process and 150 g of this product was used in the enteric coating step.

Enteric Coating of Sub-Coated Beads

Two types of subcoated beads were available for final enteric coating process and were coated with two different enteric polymers as follows:
1. Sugar sphere based beads, subcoated with Opadry II, enteric coated with Acryl EZE (methacrylic acid co-polymer type C): Formulation A.
2. Celphere based beads, subcoated with Opadry II enteric coated with Spectracyl L100 (methacrylic acid co-polymer type A): Formulation B.
   Formulation A

Enteric coating suspension was prepared by suspending Acryl EZE polymer in water to form a 20% w/w suspension. Purified water was weighed in a suitable container and was stirred vigorously to obtain a vortex. Acryl EZE powder was added slowly to ensure efficient dispersion. The dispersion was allowed to stir for at least 20 minutes before use.

Formulation B

93.75 g of Spectracyl L100 was added to 1334.38 g of isopropyl alcohol and the mixture was stirred vigerously for about 60 minutes to prevent any clumping.

78.13 g of purified water was added to this solution and a clear viscous solution resulted. 9.38 g of triethylcitrate was added to this solution and allow to stir for 10 minutes followed by the addition of 46.88 g of talc to form the final coating suspension.

Both formulations (A and B) were coated with the respective enteric coating polymer by fluid bed processing to provide a targeted 100% weight gain. A summary of the fluid bed processing parameters is provided in Table 4, below. The overall composition of Formulations A and B is provided in Table 5, below.

TABLE 5

| Processing Parameter | Formulation A | Formulation B |
|---|---|---|
| Inlet Air (SCFM) | 9-10 | 9 |
| Inlet Temp (° C.) | 39.9-51.0 | 57.1-63.5 |
| Outlet Temp (° C.) | 25.1-32.0 | 29.4-33.6 |
| Product Temp (° C.) | 29.0-32.5 | 37.8-42.9 |
| Spray Air (PSI) | 25 | 25 |
| Pump setting (%) | 5-8 | 10-100 (small tubing) |
| (rate g/min) | (1.5-2.6) | (1.6-5.2 g/min) |
| | | 25-33 (large tubing) |
| | | (6.5-8.4 g/min) |
| Filter Blow Back (PSI) | 40-65 | 30-40 |

| Formulation A | | Formulation B | |
|---|---|---|---|
| Ingredient | % | Ingredient | % |
| Ilaprazole | 12.59 | Ilaprazole | 12.59 |
| Sugar Spheres, NF (35/45) | 22.71 | Celphere CP 305, NF | 22.71 |
| Hydroxypropylcellulose | 4.07 | Hydroxypropylcellulose | 4.07 |
| (Klucel EF) | | (Klucel EF) | |
| Magnesium carbonate | 4.07 | Magnesium carbonate | 4.07 |
| Low substituted | 2.03 | Low substituted | 2.03 |
| hydroxypropylcellulose (L- | | hydroxypropylcellulose | |
| HPC) | | (L-HPC) | |
| Opadry II Y-30-18037 | 4.55 | Opadry II Y-30-18037 | 4.55 |
| Acryl EZE | 50.00 | Spectracyl L100 | 31.25 |
| | | Triethylcitrate | 3.13 |
| | | Talc | 15.62 |

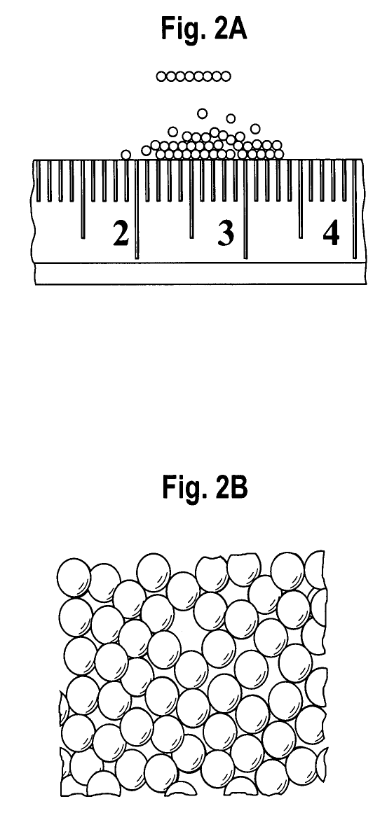

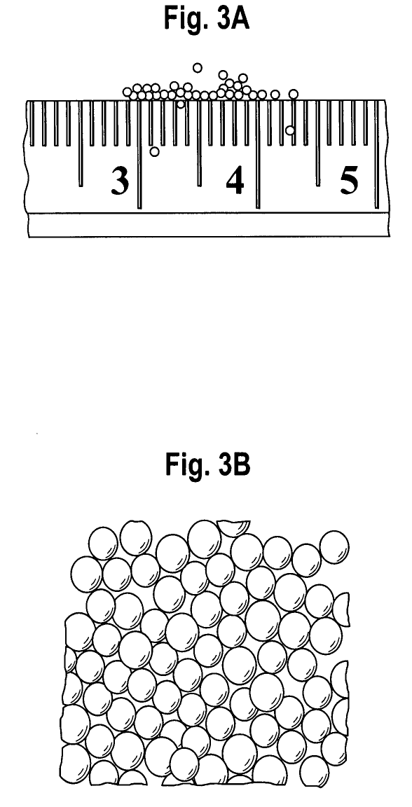

FIGS. 2 and 3 show photographs of the granules of Formulation A and B, respectively.

Capsule Filling

An appropriate quantity of granules from Formulation A was filled in size 4 capsules to provide 10 mg of ilaprazole. An appropriate quantity of granules from Formulation B was filled in size 4 capsules to provide 10 mg of ilaprazole.

Preparation of Formulation C (Enteric coated tablets)

The composition of enteric coated tablets (hereinafter referred to as “Formulation C”) is provided in Table 6, below.

TABLE 6

| Constituent | Quantity (mg) |
|---|---|
| Ilaprazole | 5.0 |
| Magnesium hydroxide | 5.0 |
| Lactose | 70.7 |
| Starch | 69.3 |
| Magnesium state | 1.0 |
| Hydroxypropyl methylcellulose 2910 (HPMC) | 4.0 |
| Titanium dioxide | 1.0 |
| Polyethylene Glycol 6000 | 1.0 |
| Hypromellose phthalate | 15.0 |
| Cetyl alcohol | 1.0 |
| Diacetylated monoglycerides | 2.0 |

Tablet Compression

Lactose and starch were blended together and granulated with starch paste. These granules were passed through a 25-mesh screen, dried at 70° C. for 5 hours, and blended with magnesium sterate for about 15 minutes. These granules were then blended with a mixture of ilaprazole (Lot B from Example 1) and magnesium hydroxide, each of which had been sieved through a 50-mesh screen (particles sieved through such a screen can have a mean particle size up to about 300 microns). This mixture was compressed into tablets.

Subcoating

Hypromellose 2910 and Polyethylene glycol 6000 was dissolved in an ethanol-water mixture (80:20). Titanium dioxide was suspended in ethanol-water mixture (80:20) and homogenized. The suspension and the solution were mixed together with stirring. This suspension was spray coated on to the uncoated tablets in a film coating processing unit with the air inlet temperature at 80° C.±5° C. and the bed temperature at 40° C.±5° C.

Enteric Coating

Hydroxypropyl methylcellulose phthalate, hypromellose and cetyl alcohol, were dissolved in a mixture of acetone/ethanol (1:1). This solution was sprayed onto the subcoated tablets in a film coating processing unit with the air inlet temperature at 75° C.±5° C. and the bed temperature at 35° C.±5° C.

### EXAMPLE 3

### Dissolution Studies with Formulations A, B and C

The purpose of this study was to determine the dissolution profiles of Formulations A, B and C. Formulations A, B and C correspond to the Formulations A, B and C described in Example 2. In this example, granules of Formulations A and B were studied. For Formulation C, 5-mg tablets were studied. A description of tablets made with Formulation C is also provided in Example 2.

Materials, Standard Preparations and Dissolution Procedure:

Materials:
Sodium Phosphate Monobasic (available from Fisher Scientific, Hampton, N.H.)

Sodium Lauryl Sulfate (hereinafter referred to as “SLS”) (available from Fisher Scientific)

Distilled Water

2N NaOH (available from Fisher Scientific)

Acetonitrile (hereinafter referred to as “CAN”), HPLC (available from Fisher Scientific)

H₂O, HPLC (available from Fisher Scientific)

Triethylamine (hereinafter referred to as “TEA”), HPLC (available from Fisher Scientific)

o-phosphoric acid, (H₃PO₄), 85% (available from Fisher)

Ilaprazole (provided by Raylo Chemicals, Inc.) hereinafter referred to as “Reference Material”)

13 mm, 0.45 um, GHP membrane filters (available from Pall Corporation, East Hills, N.Y.)

Preparation of pH 10 Diluent:
1. Mix together 1200 mL of HPLC grade water, 800 mL of HPLC grade Acetonitrile, and 20 mL of HPLC grade TEA.
2. Adjust the pH of the mixture to 10.00±0.05 with o-phosphoric acid (85%).

Preparation of Mobile Phase:
1. Mix together 1200 mL of HPLC grade water, 800 mL of HPLC grade Acetonitrile, and 20 mL of HPLC grade TEA.
2. Adjust the pH of the mixture to 7.00±0.05 with o-phosphoric acid (85%).

Preparation of pH 7.5 Buffer with 0.5% SLS (Dissolution Media):
1. Accurately weigh about 27.6 g of Sodium Phosphate Monobasic and add to a 4-L container.
2. Add 3000 mL of Distilled Water and mix well to dissolve.
3. Accurately weigh about 20 g of SLS in a 600-mL beaker and add 400 mL of Distilled Water—mix well to dissolve.
4. Add the SLS mixture to the 4-L container.
5. Use 100 mL of Distilled Water to rinse the beaker into the 4-L container.
6. Mix well and adjust the pH to 7.5±0.05 with 2N NaOH.
7. Add enough Distilled Water to bring up to 4 L total volume and mix well.
   Preparation of Standard:
1. Accurately weigh about 50 mg of Ilaprazole Reference Material and transfer it into a 100-mL volumetric flask.
2. Dilute to volume with pH 10 diluent and mix well.
3. Sonicate to dissolve the solids.
4. Further dilute 4.0 mL of the above solution to 200.0 mL with pH 10 Diluent and mix well.
5. Further dilute 25.0 mL of the above solution to 50.0 mL with pH 10 Diluent and mix well.
6. Filter a portion of the solution from step 5 above through a 13 mm, 0.45 um GHP membrane—discard the first 5 mL of filtrate and then fill HPLC vials.

HPLC Conditions: System 40019C (Shimadzu Corporation, Tokyo, Japan)

| | |
|---|---|
| Column | Capcell Pak, C18, 5 um, 4.6 × 250 mm, SN AD8832 |
| Column Temp | 25° C. (room temp) |
| Injection | 20 uL full loop injection with 50:50 ACN:H₂O needle |
| | wash, autosampler cooled to 5° C. |
| Detection | 237 nm |
| Mobile Phase | H₂O:ACN:TEA (2400:1600:40), pH 7.0 |
| Flow Rate | 1.25 mL/minute (approx. 1400 psi) |
| Integration | PeakSimple, cs = 0.5 in/min., Area Reject = 5, |
| | PS = 95.0, BS = 60.0 |
| Run Time | 9 minutes (peak at 6.7 minutes) |

Dissolution Test:
Apparatus: USP Apparatus 1 with 40 mesh baskets, rotation speed: 100 rpm
Dissolution Media: pH 7.5 Buffer with 0.5% SLS
Volume: 500 mL
Contact Time: 30 min.
Sampling Time: 10, 15, 20 and 30 min. and then analyzed by HPLC.
Temperature: 37 degrees C.±0.5° C.
1. For the tablets, one tablet was added to each basket. For the granules, weighed sample amounts were transferred into the baskets.
2. Added 500 mL of Dissolution Media to each vessel and allowed to equilibrate to 37.0±0.5° C.
3. Added the tablets/granules to a basket, attached to the shafts, lowered into the vessels, started rotation and a timer.
4. Pulled samples at 10, 15, 20, and 30 minutes using 10-mL disposable syringes and stainless steel canulas. Removed 10 mL from the vessel, replaced canula with a 13 mm, 0.45 um, GHP membrane, discarding the first 2 mL of filtrate to waste and collecting the rest in a glass test tube. Pulls were made midway between top and bottom of basket due to the low media volume.
5. Further diluted 5.0 mL of the filtrate to 10.0 mL with pH 10 Diluent and mixed well (performed immediately after samples were pulled).

Results:

A summary of the dissolution profile for each of Formulations A, B and C resulting from the in vitro dissolution test described above is shown below in Table 7. The results clearly demonstrate that at least 70% of ilaprazole in Formulations A and B is released within twenty (20) minutes when tested in the above described in vitro dissolution test. In contrast, less than 70% of ilaprazole in Formulation C is released within twenty (20) minutes when tested in the above described in vitro dissolution test.

TABLE 7

| | 10 min. | 15 min. | 20 min. | 30 min. |
|---|---|---|---|---|
| Formulation C | | | | |
| Run 1 | 33 | 59 | 66 | 72 |
| Run 2 | 18 | 59 | 65 | 71 |
| Average | 25.76 | 59.17 | 65.19 | 71.47 |
| StDev | 10.63085 | 0.18344 | 0.51762 | 0.36024 |
| RSD | 41.28 | 0.31 | 0.79 | 0.50 |
| Formulation A | | | | |
| Run 1 | 64 | 97 | 94 | 90 |
| Run 2 | 64 | 96 | 93 | 89 |
| Average | 64.15 | 96.42 | 93.70 | 89.78 |
| StDev | 0.06567 | 1.15341 | 1.10580 | 0.95466 |
| RSD | 0.10 | 1.20 | 1.18 | 1.06 |
| Formulation B | | | | |
| Run 1 | 0 | 46 | 91 | 90 |
| Run 2 | 3 | 64 | 93 | 93 |
| Average | 1.59 | 54.89 | 92.11 | 91.36 |
| StDev | 2.25057 | 12.17778 | 1.25477 | 1.79350 |
| RSD | 141.42 | 22.18 | 1.36 | 1.96 |

### EXAMPLE 4

### Bioavailability Studies with Formulations A, B and C in Dogs

The objective of this study was to assess the bioavailability of a single 10 mg oral dose of ilaprazole as delayed-release capsules relative to delayed-release tablets in male and female beagle dogs.

The formulations tested in the female beagle dogs are shown below in Table 8. In Table 8, Formulation A corresponds to Formulation A described and made in Example 2. Formulation B corresponds to Formulation B described and made in Example 2. The filling of Formulations A and B into capsules is also described in Example 2. Formulation C corresponds to Formulation C described and made in Example 2. A description of tablets made with Formulation C is also provided in Example 2.

TABLE 8

| | | | pH of Drug |
|---|---|---|---|
| Formulation | Dosage Form | API | Release |
| A | Multiparticulate | Micronized | 5.5 |
| | in Capsules | | |
| B | Multiparticulate | Micronized | 6.0 |
| | in Capsules | | |
| C | Tablet | Non | 5.2 |
| | | Micronized | |

This study was a single-dose, randomized crossover study involving 6 dogs that received a single 10 mg oral dose of ilaprazole as delayed release capsules or tablets. The group designations and dose levels are presented in Table 9, below.

TABLE 9

| | Number | | | Target Dose | Dose |
|---|---|---|---|---|---|
| Group/ | of | Dose | Dose | Level | (Capsule or |
| Phase | Animals | Formulation | Route | (mg) | Tablet) |
| 1/1 | 1 M, 1 F | Formulation A | Oral | 10 | 1 Capsule |
| 2/1 | 1 M, 1 F | Formulation B | Oral | 10 | 1 Capsule |
| 3/1 | 1 M, 1 F | Formulation C | Oral | 10 | 2 Tablets |
| 1/2 | 1 M, 1 F | Formulation B | Oral | 10 | 1 Capsule |
| 2/2 | 1 M, 1 F | Formulation C | Oral | 10 | 2 Tablets |
| 3/2 | 1 M, 1 F | Formulation A | Oral | 10 | 1 Capsule |
| 1/3 | 1 M, 1 F | Formulation C | Oral | 10 | 2 Tablets |
| 2/3 | 1 M, 1 F | Formulation A | Oral | 10 | 1 Capsule |
| 3/3 | 1 M, 1 F | Formulation B | Oral | 10 | 1 Capsule |

M Male.
F Female.
Note:
There was a washout period of at least 5 days between phases

Animals were fasted overnight prior to dosing through approximately 4 hours postdose. Prior to dose administration, each dog was pretreated with an intramuscular (“IM”) injection of pentagastrin. Pentagastrin (Sigma, St. Louis, Mo.) was dissolved in saline at a concentration of 0.25 mg/mL. One-half hour prior to the administration of each ilaprazole formulation, the dogs received a 6 μg/kg (0.024 mL/kg) IM injection of the pentagastrin solution. Individual pentagastrin doses were calculated based on body weights recorded on the day of dose administration. The capsules and tablets were given orally. Following dose administration, each dog was given approximately 30 mL of water.

In each period, venous blood samples for the determination of ilaprazole plasma concentrations were collected in tubes containing potassium EDTA prior to dosing and at 0.5, 1, 1.5, 2, 4, 6, 8, 12 and 24 hours after dose.

Plasma samples were analyzed for ilaprazole using an LC/MS/MS method for the determination of racemic ilaprazole in beagle dog plasma. The lower limit of quantitation was 5.00 ng/mL with a 0.1 mL aliquot.

Pharmacokinetic parameters for ilaprazole were determined using standard noncompartmental methods and included the observed peak plasma concentration (Cₘₐₓ), the time to reach the observed peak concentration (tₘₐₓ), the half-life of the terminal elimination phase (t_{1/2z}), and the area under the plasma concentration-time curve from time zero to the last quantifiable concentration (AUCₜ) and from time zero to infinity (AUC_{∞}). Relative bioavailability (%) was determined by dividing the AUC_{∞} of Formulation A or B by the corresponding AUC_{∞} of Formulation C.

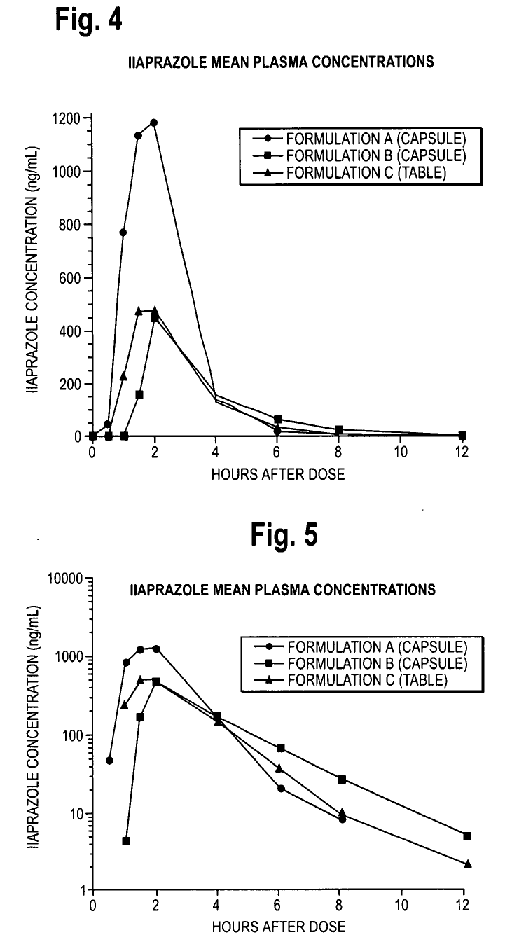

The mean pharmacokinetic values for Formulations A, B and C are summarized in Table 10 and FIG. 4.

TABLE 10

| | | tₘₐₓ | Cₘₐₓ | AUCₜ | AUC_{∞} | t_{1/2z}^{a} | Relative BA^{b} |
|---|---|---|---|---|---|---|---|
| Regimen | | (h) | (ng/mL) | (ng · h/mL) | (ng · h/mL) | (h) | (%) |
| Formulation A | N | 6 | 6 | 6 | 6 | 6 | 5 |
| | Mean | 1.58 | 1489 | 3052 | 3063 | 0.68 | 211 |
| | % CV | 24 | 25 | 42 | 42 | 18 | 47 |
| Formulation B | N | 5 | 5 | 5 | 5 | 5 | 4 |
| | Mean | 2.80 | 580 | 1408 | 1429 | 1.17 | 107 |
| | % CV | 64 | 59 | 42 | 41 | 47 | 61 |
| Formulation C | N | 5 | 5 | 5 | 5 | 5 | — |
| | Mean | 1.70 | 660 | 1495 | 1518 | 1.02 | — |
| | % CV | 16 | 47 | 35 | 35 | 25 | — |

^{a}Harmonic mean
^{b}Relative Bioavailability = (AUC_{∞} Formulation A or B/AUC_{∞} Formulation C) * 100

The objective of this study was to compare the pharmacokinetics of ilaprazole enteric-coated granules in capsule Formulations A and B to enteric-coated tablet Formulation C. The bioavailability of ilaprazole from Formulations A and B relative to Formulation C was estimated by comparing the AUC_{∞} values of each dog following administration of each formulation. As shown in Table 10, the bioavailability of Formulation A was 211% as compared to Formulation C. The bioavailability of Formulation B was 107% as compared to Formulation C. These results suggest that in pentagastrin-treated dogs, ilaprazole from Formulation A was approximately twice as bioavailable as ilaprazole from either Formulation B or C. Similar differences were observed for ilaprazole Cₘₐₓ. Values for Formulation A were more than twice as high as Cₘₐₓ values for Formulations B and C. Although Formulation A had the highest bioavailability and the highest Cₘₐₓ, its half-life was the shortest among the three formulations.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

What is claimed is:

1. A pharmaceutical composition comprising an active ingredient, wherein said active ingredient has a mean particle size of from about 0.1 micron to about 100 microns.

2. The pharmaceutical composition of claim 1, wherein said active ingredient is a compound having the following formula I: 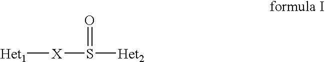
wherein Het₁ is 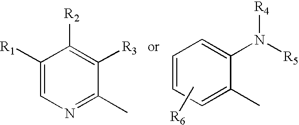
Het₂ is 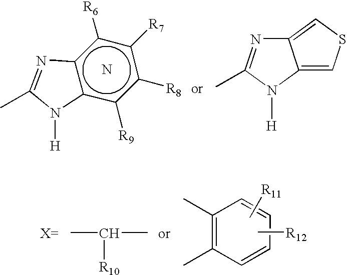
wherein
N in the benzimidazole moiety means that one of the ring carbon atoms substituted by R₆-R₉ optionally may be exchanged for a nitrogen atom without any substituents;
R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;
R₄ and R₅ are the same or different and selected from hydrogen, alkyl and arylalkyl;
R₆′ is hydrogen, halogen, trifluoromethyl, alkyl or alkoxy;
R₆-R₉ are the same or different and selected from hydrogen, alkyl, alkoxy, halogen, haloalkoxy, alkylcarbonyl, alkoxycarbonyl, oxazolinyl, trifluoroalkyl, a heterocyclic ring that may be further substituted or adjacent groups R₆-R₉ form ring structures which may be further substituted;
R₁₀ is hydrogen or forms an alkylene chain together with R₃ and R₁₁; and
R₁₂ are the same or different and selected from hydrogen, halogen or alkyl.

3. The pharmaceutical composition of claim 1, wherein said active ingredient has a mean particle size of from about 0.5 microns to about 75 microns.

4. The pharmaceutical composition of claim 3, wherein the active ingredient has a mean particle size of from about 0.75 microns to about 65 microns.

5. The pharmaceutical composition of claim 4, wherein the active ingredient has a mean particle size of from about 1 micron to about 50 microns.

6. The pharmaceutical composition of claim 1, wherein the active ingredient has a mean particle size less than about 50 microns.

7. The pharmaceutical composition of claim 6, wherein the active ingredient has a mean particle size less than about 45 microns.

8. The pharmaceutical composition of claim 7, wherein the active ingredient has a mean particle size less than about 40 microns.

9. The pharmaceutical composition of claim 1, wherein the active ingredient is 2-[[(4-methoxy-3-methyl)-2-pyridyl]methylsufinyl]-5-(1H-pyrrol-1-yl)benzimidazole.

10. The pharmaceutical composition of claim 1, wherein the active ingredient is (−)-2-[[(4-methoxy-3-methyl)-2-pyridyl]methylsufinyl]-5-(1H-pyrrol-1-yl)benzimidazole.

11. The pharmaceutical composition of claim 1, wherein the active ingredient is (+)-2-[[(4-methoxy-3-methyl)-2-pyridyl]methylsufinyl]-5-(1H-pyrrol-1-yl)benzimidazole.

12. The pharmaceutical composition of claim 1, wherein said composition further comprises at least one of a stabilizer, a surfactant, a coating, a binder, a glidant, a solubility enhancing agent, a sweetness and/or flavoring agent, a filler, lubricant, preservative, a buffer, a wetting agent, a humectant, an emulsifier, a preservative, an effervescent agent, a solution retarder, an absorption accelerator, a distintegrant or combinations thereof.

13. The pharmaceutical composition of claim 12, wherein the at least one stabilizer is a salt of a Group IA metal, a Group IIA metal, a bicarbonate salt of a Group IA metal, a bicarbonate salt of a Group IIA metal, a sodium salt, a magnesium salt, a calcium salt, an aluminum salt, a bicarbonate salt of magnesium, a bicarbonate salt of calcium, a bicarbonate salt of aluminum, polymers, sodium alginate, sterols, fatty alcohols or combinations thereof.

14. The pharmaceutical composition of claim 1, wherein said composition further comprises an enteric coating.

15. The pharmaceutical composition of claim 1, wherein said composition is a granule, microparticulate or microparticle.

16. The pharmaceutical composition of claim 15, wherein the granule, microparticulate or microparticle is placed into a capsule.

17. A method of treating a gastrointestinal disorder in a patient in need of treatment thereof, the method comprising the steps of:
administering to said patient a therapeutically effective amount of a pharmaceutical composition of claim 1.

18. The method of claim 17, wherein the gastrointestinal disorder is heartburn, inflammatory bowel disease, Crohn's disease, irritable bowel syndrome, ulcerative colitis, a peptic ulcer, a stress ulcer, a bleeding peptic ulcer, a duodenal ulcer, infectious enteritis, colitis, diverticulitis, gastric hyperacidity, dyspepsia, gastroparesis, Zollinger-Ellison syndrome, gastroesophageal reflux disease, Helicobacter pylori associated disease, short-bowel syndrome, hypersecretory states associated with systemic mastocytosis or basophilic leukemia or hyperhistaminemia or combinations of any of the above disorders.

19. The method of claim 18, wherein the gastroesophageal reflux disease is symptomatic gastroesophageal reflux disease or asymptomatic gastroesophageal reflux disease.

20. A method of treating chronic cough in a patient in need of treatment thereof, the method comprising the steps of:
administering to said patient a therapeutically effective amount of a pharmaceutical composition of claim 1.

21. A pharmaceutical composition comprising an active ingredient, wherein said active ingredient has a mean particle size of from about 0.1 micron to about 100 microns and further wherein the active ingredient is 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof.

22. The pharmaceutical composition of claim 21, wherein said active ingredient has a mean particle size of from about 0.5 microns to about 75 microns.

23. The pharmaceutical composition of claim 22, wherein the active ingredient has a mean particle size of from about 0.75 microns to about 65 microns.

24. The pharmaceutical composition of claim 23, wherein the active ingredient has a mean particle size of from about 1 micron to about 50 microns.

25. The pharmaceutical composition of claim 21, wherein the active ingredient has a mean particle size less than about 50 microns.

26. The pharmaceutical composition of claim 25, wherein the active ingredient has a mean particle size less than about 45 microns.

27. The pharmaceutical composition of claim 26, wherein the active ingredient has a mean particle size less than about 40 microns.

28. The pharmaceutical composition of claim 21, wherein the active ingredient is 2-[[(4-methoxy-3-methyl)-2-pyridyl]methylsufinyl]-5-(1H-pyrrol-1-yl)benzimidazole.

29. The pharmaceutical composition of claim 21, wherein the active ingredient is (−)-2-[[(4-methoxy-3-methyl)-2-pyridyl]methylsufinyl]-5-(1H-pyrrol-1-yl)benzimidazole.

30. The pharmaceutical composition of claim 21, wherein the active ingredient is (+)-2-[[(4-methoxy-3-methyl)-2-pyridyl]methylsufinyl]-5-(1H-pyrrol-1-yl)benzimidazole.

31. The pharmaceutical composition of claim 21, wherein said composition further comprises at least one of a stabilizer, a surfactant, a coating, a binder, a glidant, a solubility enhancing agent, a sweetness and/or flavoring agent, a filler, lubricant, preservative, a buffer, a wetting agent, a humectant, an emulsifier, a preservative, an effervescent agent, a solution retarder, an absorption accelerator, a distintegrant or combinations thereof.

32. The pharmaceutical composition of claim 31, wherein the at least one stabilizer is a salt of a Group IA metal, a Group IIA metal, a bicarbonate salt of a Group IA metal, a bicarbonate salt of a Group IIA metal, a sodium salt, a magnesium salt, a calcium salt, an aluminum salt, a bicarbonate salt of magnesium, a bicarbonate salt of calcium, a bicarbonate salt of aluminum, polymers, sodium alginate, sterols, fatty alcohols or combinations thereof.

33. The pharmaceutical composition of claim 21, wherein said composition further comprises an enteric coating.

34. The pharmaceutical composition of claim 21, wherein said composition is a granule, microparticulate or microparticle.

35. The pharmaceutical composition of claim 34, wherein the granule, microparticulate or microparticle is placed into a capsule.

36. The pharmaceutical composition of claim 21, wherein at least 70% of the 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof is released from the composition within about 20 minutes when tested in an in vitro dissolution test Apparatus 1 (basket method) in 500 mL of a pH 7.5 buffer with about 0.5 sodium lauryl sulfate as the dissolution medium, at about 100 rpm and at a temperature of about 37° C.±0.5° C.

37. The pharmaceutical composition of claim 21, wherein at least 75% of the 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof is released from the composition within about 20 minutes when tested in an in vitro dissolution test Apparatus 1 (basket method) in 500 mL of a pH 7.5 buffer with about 0.5 sodium lauryl sulfate as the dissolution medium, at about 100 rpm and at a temperature of about 37° C.±0.5° C.

38. The pharmaceutical composition of claim 21, wherein at least 80% of the 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals, or combinations thereof is released from the composition within about 20 minutes when tested in an in vitro dissolution test Apparatus 1 (basket method) in 500 mL of a pH 7.5 buffer with about 0.5 sodium lauryl sulfate as the dissolution medium, at about 100 rpm and at a temperature of about 37° C.±0.5° C.

39. The pharmaceutical composition of claim 21, wherein at least 85% of the 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof is released from the composition within about 20 minutes when tested in an in vitro dissolution test Apparatus 1 (basket method) in 500 mL of a pH 7.5 buffer with about 0.5 sodium lauryl sulfate as the dissolution medium, at about 100 rpm and at a temperature of about 37° C.±0.5° C.

40. The pharmaceutical composition of claim 21, wherein at least 90% of the 2-[[(4-methoxy-3-methyl)-2-pyridyl]methylsufinyl]-5-(1H-pyrrol-1-yl)benzimidazole, (−)-2-[[(4-methoxy-3-methyl)-2-pyridyl]methylsufinyl]-5-(1H-pyrrol-1-yl)benzimidazole, (+)-2-[[(4-methoxy-3-methyl)-2-pyridyl]methylsufinyl]-5-(1H-pyrrol-1-yl)benzimidazole, salts, metabolites, polymorphs, cocrystals or combinations thereof is released from the composition within about 20 minutes when tested in an in vitro dissolution test Apparatus 1 (basket method) in 500 mL of a pH 7.5 buffer with about 0.5 sodium lauryl sulfate as the dissolution medium, at about 100 rpm and at a temperature of about 37° C.±0.5° C.

41. A method of treating a gastrointestinal disorder in a patient in need of treatment thereof, the method comprising the steps of:
administering to said patient a therapeutically effective amount of a pharmaceutical composition of claim 21.

42. The method of claim 41, wherein the gastrointestinal disorder is heartburn, inflammatory bowel disease, Crohn's disease, irritable bowel syndrome, ulcerative colitis, a peptic ulcer, a stress ulcer, a bleeding peptic ulcer, a duodenal ulcer, infectious enteritis, colitis, diverticulitis, gastric hyperacidity, dyspepsia, gastroparesis, Zollinger-Ellison syndrome, gastroesophageal reflux disease, Helicobacter pylori associated disease, short-bowel syndrome, hypersecretory states associated with systemic mastocytosis or basophilic leukemia or hyperhistaminemia or combinations of any of the above disorders.

43. The method of claim 42, wherein the gastroesophageal reflux disease is symptomatic gastroesophageal reflux disease or asymptomatic gastroesophageal reflux disease.

44. A method of treating chronic cough in a patient in need of treatment thereof, the method comprising the steps of:
administering to said patient a therapeutically effective amount of a pharmaceutical composition of claim 21.
